(19) United States
(12) Patent Application Publication
(10) Pub. No.: US 2008/0207778 A1
(43) Pub. Date: Aug. 28, 2008

(54) Support Comprising a Wax Derivative for Making Topical Cosmetic Formulations

(75) Inventors: Jean-David Rodier, Villeurbanne (FR); Vincent Hubiche, Saint Laurent De Mure (FR); Bruno Mahler, Lyon (FR)

Correspondence Address: CANTOR COLBURN, LLP, 20 Church Street, 22nd Floor, Hartford, CT, 06103, US

(73) Assignee: GATTEFOSSE SAS, Saint-Priest (FR)

(21) Appl. No.: 12/090,189

(22) PCT Filed: Oct. 9, 2006
(86) PCT No.: PCT/FR2006/051007

§ 371 Date: Apr. 14, 2008

(30) Foreign Application Priority Data
Oct. 12, 2005 (FR) 0553101

(51) Int. Cl. A61K 47/14; A61Q 99/00

(52) U.S. Cl. 514/787; 514/785

(57) ABSTRACT

Disclosed is a support, in particular for making topical cosmetic formulations comprising a mixture containing polyol esters with saturated linear acids and unsaturated linear and/or saturated branched acids, monounsaturated linear or saturated branched acid esters with saturated linear alcohols, saturated linear acid esters with monounsaturated linear or saturated branched alcohols, saturated linear and/or monounsaturated linear and/or saturated branched free alcohols, saturated linear ester acids and saturated linear alcohols, monounsaturated linear or saturated branched ester acids and monounsaturated linear or saturated branched linear alcohols, the linear saturated alcohols and acids having at least 20 carbon atoms and the monounsaturated or saturated branched alcohols and acids having at least 18 carbon atoms.

The invention relates to a support comprising a wax derivative, for making in particular topical cosmetic formulations. In practice, the support of the invention can be used in cosmetics and pharmaceuticals, in particular for its emulsifying effect according to the cases of W/O or O/W, and also for its softening effect. The invention is more particularly described below in relation to the cosmetic field, although it can be used, as already stated, in the pharmaceutical field.

Waxes are widely used in cosmetics, in particular as hardeners in lipsticks or as thickeners in certain emulsions. Their composition is particularly complex. They have the common feature of containing a mixture of acid monoesters and very long chain fatty alcohols.

A distinction is made between solid waxes and liquid waxes.

Solid waxes are waxes whereof the melting point is between 50 and 90° C. They correspond to mixtures essentially comprising monoesters having the formula R¹—C(O)—O—R², where the R¹—C(O)— group corresponds to the carbon chain of the fatty acid, the said acid usually being linear and saturated and having a number of carbon atoms equal to 20 or higher. This therefore includes the acid comprising at least 20 carbon atoms, that is eicosanoic (or arachidic) acid, the C22, docosanoic (or behenic), C24, tetracosonoic (or lignoceric), C26 hexacosonic (or cerotic), up to C34 acids. Depending on the source of the wax, the mixture of monoesters may also contain a certain proportion of hydroxyacid esters such as hydroxypalmitic (C16), or hydroxystearic acid. This is the case for example of beeswax. The R² group corresponds to the hydrocarbon chain of the alcohol, the said alcohol usually being saturated linear and having a number of carbon atoms equal to 20 or higher. It may therefore concern C20 eicosanol, C22 docosanol, C24 tetracosanol and so on up to C34. Depending on the source of the wax, the mixture of monoesters may also contain a certain proportion of diol esters. This is the case for example of carnauba wax.

Beeswax, carnauba wax, candelilla wax, rice wax and sugarcane wax are examples of natural solid waxes.

Liquid waxes are mixtures of acid monoesters and very long chain fatty alcohols being present in the liquid state at ambient temperature, or a temperature of between 15° C. and about 25° C. These monoesters have the formula R³—C(O)—O—R⁴, where the R³—C(O) group corresponds to the carbon chain of the fatty acid. This chain may be linear or monounsaturated. It comprises a number of carbon atoms equal to 18 or higher. Mention can be made of oleic (C18:1), gadoleic (C20:1), erucic (C22:1) acid, up to hexaconenoic (C26:1) acid for unsaturated acids. The R³—C(O) group may also consist of branched and saturated acids having a number of carbon atoms higher than C18, also called Guerbet acids. The R⁴—O— group may consist of monounsaturated linear fatty alcohols with a number of carbon atoms equal to 18 or higher. Mention can therefore be made of C18 octadecenol, C20 eicosenol, C22 docosenol up to C26, hexacosenol. The carbon chain of the alcohol may also be branched and saturated and comprise a number of carbon atoms equal to C18 or higher. Such alcohols are also called Guerbet alcohols. A liquid wax known today is jojoba wax, commonly called jojoba oil, the liquid nature being due to the presence of monounsaturated chains.

Document U.S. Pat. No. 5,660,865 describes the use in cosmetics of a mixture of solid waxes and liquid waxes without addition of polyol.

Document U.S. Pat. No. 6,630,134 B1 describes the cosmetic use of esters of solid beeswax or candelilla wax or carnauba wax with a Guerbet alcohol. The three products obtained from the three waxes are used in hair gels.

Document U.S. Pat. No. 6,432,428 B1 describes the use in cosmetics, particularly as softening agent, of derivatives of jojoba and hydrogenated jojoba wax obtained by alcoholysis by isoproplyl alcohol.

Document EP 795 317 B1 describes a cosmetic composition for treating the skin or hair comprising a combination of waxes consisting of soft wax such as apple wax and orange wax and a liquid wax, in the present case jojoba wax.

Document U.S. Pat. No. 6,258,965 describes the reaction product between a wax and meadowsweet (Limnanthes Alba) seed oil, also called meadowfoam oil, corresponding to a triglyceride containing C20 and C22 long chain monounsaturated fatty acids. In practice, an interesterification reaction occurs between the fatty acids of the triglycerides and the fatty alcohols consisting of the wax.

Document U.S. Pat. No. 6,706,768 B2 describes a composition obtained by saponification, followed by alloxylation of a mixture of hydrogenated and non-hydrogenated jojoba wax in the absence of polyol. The mixture obtained contains ethers.

To the knowledge of the Applicant, also available on the market are mixtures of solid and liquid waxes separately modified with polyols. Mention can be made for example of jojoba wax PEG-80 Esters (Florasolv® from Floratech), PEG-8 Beeswax (product of the reaction between beeswax and PEG) (Apifil® from Gattefossé) mentioned in document EP 1 286 679 B1, Bis-PEG-12 Dimethicone Beeswax (Siliconyl Beeswax from Koster Keunen), Polyglyceryl-3 Beeswax (Jan Dekker and Koster Keunen) mentioned in document U.S. Pat. No. 5,387,579.

The Applicant has found very unexpectedly that the product of the simultaneous reaction between a solid wax, a liquid wax and a polyol, had a lower hardness than the product issuing from the mixture of the two reaction products previously described, respectively a solid wax modified by a polyol and a liquid wax modified by the same polyol. This different behaviour could, according to the Applicant, result from the presence of a novel polyol derivative which is nonexistent when the products are mixed separately, the said polyol derivative corresponding to a structure in which all or part of the OH functions of the polyol are esterified with the saturated linear fatty acids of the solid wax and the unsaturated linear or saturated branched fatty acids of the liquid wax. Moreover, this transesterification reaction causes a random redistribution of the acids and alcohols constituting the waxes. At the end of the reaction, the wax derivative therefore contains monounsaturated linear or saturated branched acid esters and saturated linear alcohols, saturated linear acid esters and monounsaturated linear or saturated branched alcohols, saturated linear free alcohols, monounsaturated linear and/or saturated branched free alcohols and the polyol esters previously described, in addition to the still unreacted starting constituents.

In other words, the invention relates to a support, in particular for malting topical cosmetic formulations comprising a mixture essentially containing:
polyol esters with saturated linear acids and unsaturated linear and/or branched acids,
monounsaturated linear or saturated branched acid esters with saturated linear alcohols,
saturated linear acid esters with monounsaturated linear or saturated branched alcohols,
saturated linear and/or monounsaturated linear and/or saturated branched free alcohols,
saturated linear ester acids and saturated linear alcohols,
monounsaturated linear or saturated branched ester acids and monounsaturated linear or saturated branched linear alcohols,
   the linear saturated alcohols and acids having at least 20 carbon atoms and the monounsaturated or branched alcohols and acids having at least 18 carbon atoms.

In a first embodiment, the mixture is a wax derivative obtained by reacting together at least one solid wax and at least one liquid wax in the presence of at least one polyol and at least one catalyst. As already stated, a transesterification reaction occurs between the various chemical entities yielding the above product.

In practice, the solid wax is a natural wax advantageously selected from the group comprising carnauba wax, candelilla wax, rice bran wax, sunflower wax, sugarcane wax, ouricury wax, beeswax, Shellac wax.

Similarly, the liquid wax is a natural wax, in practice jojoba wax. This wax comprises in particular C18:1 (minority), C20:1 and C22:1 (majority with C20:1>C22:1) unsaturated fatty acid esters, with C20:1 and C22:1 then C24:1 unsaturated fatty alcohols.

In a second embodiment, the mixture is obtained by reacting saturated linear acids and alcohols with monounsaturated linear and/or saturated branched linear acids and alcohols, and polyols, in the presence or absence of a catalyst, the saturated linear acids and alcohols having at least 20 carbon atoms and the monounsaturated linear or saturated branched acids and alcohols having at least 18 carbon atoms. An esterification reaction occurs yielding the product of the invention.

According to another feature, the polyol is selected from the group comprising ethylene glycol, diethylene glycol, triethylene glycol, 2-methyl propanediol, propylene glycol, butylene glycol, neopentyl glycol, hexylene glycol, octylene glycol, polyethylene glycol, polypropylene glycol, trimethylol propane, sorbitol, erythritol, pentaerythritol, dipentaerythritol, glycerol, diglycerol, polyglycerol.

When the support is prepared from wax, the presence of polyol in the reaction confers a certain hydrophilic property to the wax derivative, and hence surface-active properties. The incorporation of a polyol increases the free OH content in the final product. The O/W or W/O surface-active behaviour of the derivative varies according to the type and proportion of the polyol employed.

A wax derivative obtained mainly from liquid wax, solid wax and polyglycerol has water-in-oil emulsifying behaviour as well as a capacity to absorb water as naturally observed in lanoline.

In practice, the proportion of esterified polyol represents between 0.5 and 50% by weight of the mixture, the proportion of esterified fatty acids represents between 20 and 60% by weight of the mixture and the proportion of esterified fatty alcohols between 20 and 60% by weight of the mixture.

The molecular conversions occurring during the transesterification reaction are obtained in the presence of catalysts. The preferred catalysts are alkaline or alkaline earth hydroxides or alkoxides, such as caustic soda and potash, in alcohol solution, aqueous or in solid form, calcium hydroxide, potassium or sodium carbonates, catalysts based on tin or titanium.

In a preferred embodiment, the wax derivative is obtained by reacting jojoba wax, rice wax, candelilla wax and polyglycerol.

In practice, the reaction is conducted at a temperature of between 100° C. and 220° C., advantageously between 150 and 200° C.

Depending on the desired properties of the support, the liquid wax/solid wax mass proportion, like the wax/polyol mass proportion, varies.

In practice, the liquid wax/solid wax mass ratio varies between 5/95 and 95/5, advantageously between 30/70 and 75/25. The wax/polyol mass ratio varies between 1/99 and 99/1, advantageously between 95/5 and 50/50.

The support of the invention has water-in-oil and oil-in-water emulsifying properties, according to the polyol selected. For example, the use of polyglycerol serves to obtain a lipophilic emulsifier producing water-in-oil emulsions with regard to the capacity of the derivative obtained to absorb large quantities of water. On the contrary, the use of PEG for example serves to obtain a hydrophilic emulsifier producing oil-in-water emulsions.

Accordingly, a further object of the invention is the use of W/O or O/W emulsifier, of the support previously described.

In a particular embodiment, the support of the invention, when used as emulsifier, contains at least the wax derivative previously described and up to advantageously three of the constituents selected from the group of sodium stearoyl lactylate, cetostearyl alcohol, glycerol stearate.

Advantageously, the support is an emulsifying system consisting of a wax derivative produced by the reaction between jojoba wax, rice bran wax, candelilla wax, in the presence of polyglycerol and of the four preceding constituents (4-50% sodium stearoyl lactylate, 4-50% cetostearyl alcohol, 4-50% glycerol stearate, 4-50% wax derivative by weight of the weight of the support).

A further subject of the invention is a cosmetic composition comprising the support previously described.

When the support consists of a wax derivative previously described, the wax derivative represents between 0.01 and 99% by weight, preferably between 0.1 and 10% by weight of the cosmetic composition.

The support of the invention is generally applied topically. It also has sensory properties of softness when applied and thereafter, which are particularly appreciated.

The support of the invention in general, and the wax derivatives in particular, may be formulated in all galenic forms normally used for a topical application to the skin or hair, in particular in a cosmetic composition in the form of an oil-in-water or water-in-oil or multiple emulsion, a silicone emulsion, a microemulsion or nanoemulsion.

The composition may be more or less fluid and have the appearance, among others, of a white or coloured crème, a pomade, a milk, a lotion, a serum.

The composition may contain the usual additives in the cosmetic and dermatological fields, such as fats, emulsifiers and co-emulsifiers, hydrophilic or lipophilic gelling agents, hydrophilic or lipophilic active ingredients, preservatives, antioxidants, solvents, perfumes, fillers, hydrophilic and lipophilic filters, dyestuffs, neutralizers, propenetrating agents, and polymers.

The quantities of these various additives are those conventionally used in the fields concerned, and for example 0.01 to 30% of the total weight of the composition. These additives, according to their nature, can be introduced in the fatty phase or in the aqueous phase.

As fatty materials usable in the cosmetic composition of the invention, use can be made of mineral oils, oils of animal origin (lanoline), vegetable oils, synthetic oils (isopropyl myristate, octyldodecyl, isostearyl isostearate, decyl oleate, isopropyl palmitate), silicone oils (cyclomethicone, dimethicone) and fluorinated oils. As fatty materials, use can be made of fatty alcohols, fatty acids, waxes and gums and in particular silicone elastomers.

As emulsifiers and co-emulsifiers usable in the cosmetic composition of the invention, mention can be made for example of esters of polyglycerols and fatty acids, esters of saccharose and fatty acids, esters of sorbitan and fatty acids, esters of fatty acids and polyoxyethylenated sorbitan, esters of fatty alcohols and PEG, esters of glycerol and fatty acids, alkylsulphates, alkyl ether sulphates, alkyl phosphates, alkyl polyglucosides, dimethicones copolyols, and derivatives of citric acid and lactic acid such as Sodium Stearoyl Lactylate.

As hydrophilic gelling agents, mention can be made in particular of carboxy vinyl polymers (carbomer), acrylic copolymers such as acrylate/alkylacrylate copolymers, polyacrylamide, polysaccharides such as xanthan gum, guar gum, natural gums such as cellulose gum and derivatives thereof, starches and derivatives thereof, clays and copolymers of 2-acrylamido-2-methylpropane acid.

As lipophilic gelling agents, mention can be made of modified clays such as bentones, metal salts of fatty acids, silica and derivatives thereof, and ethylcellulose.

The cosmetic composition may also contain active ingredients. As active ingredients, use can be made in particular of depigmenting agents, softening agents, moisturizing agents, anti-seborrheic, anti-inflammatory, anti-acne agents, keratolytic and/or desquamative agents, anti-wrinkle agents and tensors, draining agents, anti-irritants, soothing agents, slimming agents such as xanthic bases (caffeine), vitamins and mixtures thereof, matifying agents, anti-aging agents such as retinol, anti-wrinkle agents and essential oils.

As preservatives usable according to the invention, mention can be made of benzoic acid, its salts and esters; sorbic acid and salts thereof; parabens, salts and esters thereof; triclosan; imidaxolidinyl urea; phenoxyethanol; DMDM hydantoin; diazolidinyl urea; chlorphenesin.

As antioxidants usable according to the invention, mention can be made of chelating agents such as EDTA and salts thereof.

As solvents usable according to the invention, mention can be made of water, ethanol, glycerine, propylene glycol, butylene glycol, sorbitol.

As fillers usable according to the invention, mention can be made of talc, kaolinite, mica, serecite, magnesium carbonate, aluminium silicate, magnesium silicate, organic powders such as nylon.

As filters usable according to the invention, mention can be made of UVA and UVB filters conventionally used such as benzophenone-3, butyl methoxydibenzoyl methane, octocrylene, octyl methoxycinnamate, 4-methylbenzylidene camphor, octyl salicylate, tacephthalydene dicamphor sulphanic acid, and drometrizole trisiloxane. Mention can also be made of physical filters TiO₂ and ZnO in their micrometric and nanometric forms, coated or uncoated.

As dyestuffs usable according to the invention, mention can be made of lipophilic dyes, hydrophilic dyes, pigments and mother-of-pearl commonly used in cosmetic or dermatological compositions, and mixtures thereof.

As neutralizers usable according to the invention, mention can be made of caustic soda, triethanolamine, aminomethyl propanol, potassium hydroxide.

As propenetrating agents usable according to the invention, mention can be made of alcohols and glycols (ethanol, propylene glycol), ethoxydiglycol, alcohols and fatty acids (oleic acid), fatty acid esters, dimethyl isosorbide.

The composition according to the invention can be used as a care product, as a cleaning product, and/or as a make-up product for the skin, as a sun protection product, or as a hair product.

The invention and the advantages thereof will clearly appear from the exemplary embodiments below in conjunction with the appended figures.

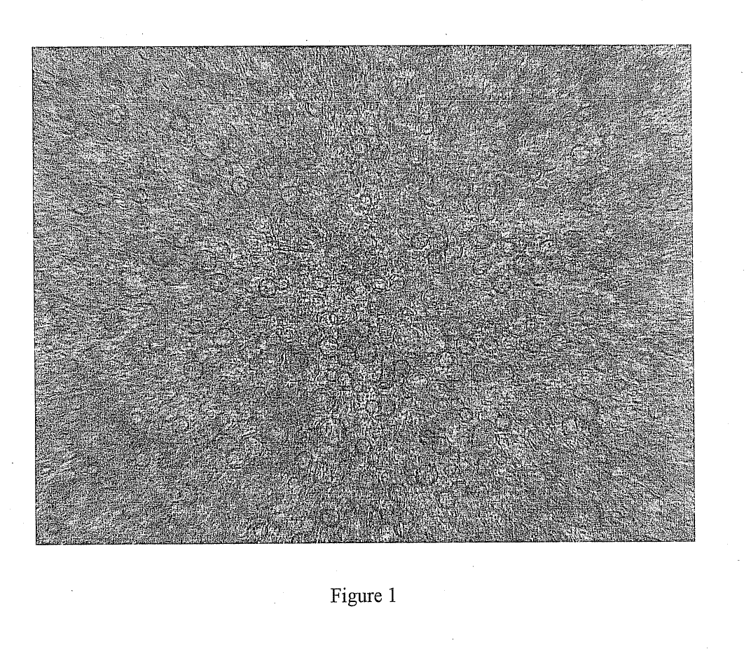

FIG. 1 is a microscopic view of an emulsion formed by the wax derivative of the invention.

### EXAMPLE 1

Production of the wax derivative in the liquid wax/solid wax mass proportion of about 70/30 and wax/polyol mass proportion of 85/15.

### Composition of the Initial Reaction Mixture

| | |
|---|---|
| Jojoba wax | 571 g |
| Candelilla wax | 123 g |
| Rice wax | 122 g |
| Triglycerol | 144 g |
| K2CO3 | 40 g |

The products are introduced into a two-litre stirred reactor. The reaction takes place at 200° C., for eight hours with nitrogen inerting. At the end of the temperature plateau, the catalyst is neutralized. The wax derivative is recovered and filtered. The quantity of wax derivative recovered is about 780 g. The product obtained is beige in colour and has a soft consistency.

The product has the capacity to form water-in-oil emulsions. A microscopic view of the emulsion reveals drops of water dispersed in the wax phase.

The invention and the advantages thereof will appear clearly from the exemplary embodiments below in conjunction with FIG. 1, which is a microscope photograph of the emulsion of example 1.

### EXAMPLE 2

Production of the wax derivative in liquid wax/solid wax proportion of 50/50 and wax/polyol proportion of 90/10.

| | |
|---|---|
| Jojoba wax | 432 g |
| Candelilla wax | 432 g |
| PEG 200 | 96 g |
| Aqueous NaOH | 40 g |

The products are introduced into a two-litre stirred reactor. The reaction takes place at 200° C., for eight hours with nitrogen inerting. At the end of the temperature plateau, the catalyst is neutralized by a sufficient quantity of citric acid. The wax derivative is then filtered. The quantity recovered is about 800 g. The product obtained is pale beige in colour and has a soft consistency.

The product has the capacity to form oil-in-water emulsions.

### EXAMPLE 3

Production of the wax derivative in the liquid wax/solid wax mass proportion of about 60/40 and wax/polyol mass proportion of 80/20.

### Composition of the Initial Reaction Mixture

| | |
|---|---|
| Jojoba wax | 461 g |
| Rice wax | 307 g |
| Propylene glycol | 192 g |
| K2CO3 | 40 g |

The products are introduced into a two-litre stirred reactor. The reaction takes place at 150° C., for eight hours with nitrogen inerting. At the end of the temperature plateau, the catalyst is neutralized by a sufficient quantity of phosphoric acid. The excess propylene glycol is removed under a pressure of 5 mbar and the wax derivative finally obtained is then filtered. The quantity of wax derivative recovered is about 680 g. The product obtained is light brown in colour and has a soft consistency.

The product thickens the cosmetic formulas and can be used as a consistency agent.

### EXAMPLE 4

Comparative hardness tests, evaluation of consistency of the wax derivative. In this example, the hardness of the wax derivative of example 1 is compared with that of product resulting from the mixture of the three derivatives of each wax prepared separately. The procedure applied to obtain the wax derivative is the one described in example 1.

Jojoba+candelilla+rice waxes+polyglycerol+catalyst→wax derivative of example 1

Jojoba wax+polyglycerol+catalyst

Candelilla wax+polyglycerol+catalyst→mixture containing each

Rice wax+polyglycerol+catalyst wax derivative

The reaction product is found to be much softer than the mixture. This characteristic can be highlighted by measuring the hardness reflected by the penetration depth of a needle applying a limited force of 2N.

Apparatus used for the hardness measurement
Chatillon make TCD 200 support
Indelco DGGS dynamometer, equipped with a needle, limited to 2.5 N.

Principle: the needle penetrates the sample. The force applied therefore increases.

The penetration depth is then measured when the force reaches 2N.

The following results are obtained:

Wax derivative: 4.4 mm
Mixture of derivatives: 2.4 mm.

The wax derivative is therefore much softer than the mixture of each wax derivative. The wax derivative of example 1 and the mixture of each derivative have the same composition of each of the waxes (candelilla, rice and jojoba).

This demonstrates the importance of the polyol derivative containing the monounsaturated and saturated acid chains, as well as the product of the reaction of the waxes together, which soften the final product.

### EXAMPLE 5

### Examples of Formulations

Anti-Wrinkle Day Crème

| Ingredients | Quantity (%) |
|---|---|
| Wax derivative of the invention according to Example 1 | 2.00 |
| Sodium stearoyl lactylate | 0.50 |
| Stearic acid | 1.00 |
| Cyclopentasiloxane | 3.00 |
| Octyldodecyle myristate | 2.00 |
| Cetostearyl alcohol | 1.00 |
| Glycerol stearate | 0.50 |
| Octyl methoxycinnamide | 5.00 |
| Benzophenone-3 | 2.00 |
| Aluminium starch octenylsuccinate | 3.00 |
| Phenoxyethanol, methylparaben, butylparaben, | 1.00 |
| ethylparaben, propylparaben | |
| Modified starch | 1.50 |
| Xanthan gum | 0.20 |
| Disodium EDTA | 0.05 |
| Glycerine | 3.00 |
| Liquid extract of Acmella Oleacera | 2.00 |
| Ethanol | 3.00 |
| Tocopherol acetate | 0.50 |
| Perfume | 0.40 |
| Water | Qsp 100 |

### Body Milk

| Ingredients | Quantity (%) |
|---|---|
| PEG-6 Stearate, Ceteth-20, steareth-20 | 8.00 |
| Propylene glycol dipelargonate | 10.00 |
| Stearic acid | 1.00 |
| Wax derivative of the invention according to Example 2 | 2.00 |
| Kernel oil | 3.00 |
| Dimethicone | 2.00 |
| Tocopherol acetate | 0.50 |
| Cyclomethicone | 3.00 |
| Phenoxyethanol, methylparaben, butylparaben, | 1.00 |
| ethylparaben, propylparaben | |
| Carbomer | 0.15 |
| Xanthan gum | 0.30 |
| Glycerine | 3.00 |
| Sodium hydroxide (10% solution) | 0.30 |
| Ascorbic acid | 0.05 |
| Perfume | 0.40 |
| Water | Qsp 100 |

### O/W Emulsion

| Ingredients | Quantity (%) |
|---|---|
| Phenoxyethanol, methylparaben, butylparaben, | 1.0 |
| ethylparaben, propylparaben | |
| Water | Qsp 100 |
| Magnesium aluminium silicate | 1.5 |
| Glycerine | 3.0 |
| Xanthan gum | 0.1 |
| Polysorbate-60 | 0.9 |
| Glyceryl stearate, PEG-100 stearate | 2.1 |
| Cetyl alcohol | 2.6 |
| Wax derivative of the invention according to Example 3 | 1.5 |
| Paraffin oil | 7.5 |
| Isopropyl myristate | 7.5 |
| Ethoxydiglycol | 5.0 |
| Perfume | 0.2 |
| Triethanolamine | 0.3 |

### W/O Emulsion

| Ingredients | Quantity (%) |
|---|---|
| Glycerine | 3.0 |
| Phenoxyethanol, methylparaben, butylparaben, | 1.0 |
| ethylparaben, propylparaben | |
| Magnesium sulphate | 0.7 |
| Cetyl dimethicone copolyol | 2.5 |
| Wax derivative of the invention according to Example 1 | 3.0 |
| Isohexadecane | 3.5 |
| Caprylic/capric triglyceride | 5.8 |
| Dimethicone | 4.0 |
| Perfume | 0.1 |
| Water | Qsp 100 |

### Multiple W/O/W Emulsion

| Ingredients | Quantity (%) |
|---|---|
| PEG-30 dipolyhydroxystearate | 2.40 |
| Isohexadecane | 9.00 |
| PPG-15 stearyl ether | 4.50 |
| Wax derivative of the invention according to Example 3 | 1.20 |
| Caprylic/capric triglyceride | 4.50 |
| Magnesium sulphate | 0.82 |
| Propylene glycol, diazolidinyl urea, methylparaben, | 1.20 |
| propylparaben | |
| Poloxamer 407 | 2.00 |
| Glycerine | 3.00 |
| Xanthan gum | 0.70 |
| Perfume | 0.20 |
| Water | Qsp 100 |

Sun Crème

| Ingredients | Quantity (%) |
|---|---|
| DEA cetyl phosphate | 2.0 |
| Glyceryl stearate, PEG-100 stearate | 4.0 |
| Wax derivative of the invention according to Example 2 | 2.0 |
| Octyl methoxycinnamate | 7.0 |
| Butyl methoxydibenzoylmethane | 2.0 |
| Benzophenone-3 | 1.0 |
| Titanium dioxide | 3.0 |
| Butylene glycol cocoate | 3.0 |
| Cyclomethicone | 2.0 |
| Tocopherol acetate | 0.5 |
| EDTA | 0.1 |
| Acrylate C/C10-30 alkyl acrylates crosspolymer | 0.2 |
| Xanthan gum | 0.3 |
| Phenoxyethanol, methylparaben, ethylparaben, | 1.0 |
| propylparaben, isobutylparaben | |
| Butylene glycol | 3.0 |
| Sodium hydroxide (10% solution) | 0.4 |
| Perfume | 0.3 |
| Water | Qsp 100 |

Foundation Crème

| Ingredients | Quantity (%) |
|---|---|
| Glyceryl stearate, propylene glycol stearate, glyceryl | 5.0 |
| isostearate, propylene glycol isostearate, oleth-25, ceteth-25 | |
| Wax derivative of the invention according to Example 3 | 4.0 |
| Glyceryl dibehenate, tribehenin, glyceryl behenate | 1.00 |
| Ethoxydiglycol oleate | 9.00 |
| Isostearyl isostearate | 5.00 |
| Cetostearyl alcohol | 2.00 |
| Dimethicone | 5.00 |
| Tocopherol acetate | 0.50 |
| Phenoxyethanol, methylparaben, ethylparaben, | 0.60 |
| propylparaben, isobutylparaben | |
| Xanthan gum | 0.40 |
| Microcrystal cellulose, cellulose gum | 1.50 |
| Titanium dioxide | 6.60 |
| Iron oxides (yellow pigment) | 1.55 |
| Iron oxides (red pigment) | 0.43 |
| Iron oxides (black pigment) | 0.11 |
| Dimethicone, dimethiconol | 3.00 |
| Water | Qsp 100 |

1. Support, in particular for making topical cosmetic formulations comprising a mixture containing:
polyol esters with saturated linear acids and unsaturated linear and/or saturated branched acids,
monounsaturated linear or saturated branched acid esters with saturated linear alcohols,
saturated linear acid esters with monounsaturated linear or saturated branched alcohols,
saturated linear and/or monounsaturated linear and/or saturated branched free alcohols,
saturated linear ester acids and saturated linear alcohols,
monounsaturated linear or saturated branched ester acids and monounsaturated linear or saturated branched linear alcohols,
the linear saturated alcohols and acids having at least 20 carbon atoms and the monounsaturated or saturated branched alcohols and acids having at least 18 carbon atoms.

2. Support according to claim 1, wherein the mixture is a wax derivative obtained by reacting together at least one solid wax and at least one liquid wax in the presence of at least one polyol.

3. Support according to claim 2, wherein the solid wax is selected from the group comprising carnauba wax, candelilla wax, rice bran wax, sunflower wax, sugarcane wax, ouricury wax, beeswax, Shellac wax.

4. Support according to claim 2, wherein the liquid wax is jojoba wax.

5. Support according to claim 2, wherein the poylol is selected from the group comprising ethylene glycol, diethylene glycol, triethylene glycol, propylene glycol, butylene glycol, neopentyl glycol, 2-methyl propanediol, hexylene glycol, octylene glycol, polyethylene glycol, polypropylene glycol, trimethylol propane, sorbitol, erythritol, pentaerythritol, dipentaerythritol, glycerol, diglycerol, polyglycerol.

6. Support according to claim 1, wherein the esterified polyol represents between 0.5 and 50% by weight of the mixture, the proportion of esterified fatty acids represents between 20 and 60% by weight of the mixture and the proportion of esterified fatty alcohols between 20 and 60% by weight of the mixture.

7. Support according to claim 2, wherein the wax derivative is obtained by reacting jojoba wax, rice wax, candelilla wax and polyglycerol.

8. Support according to one of claim 2, wherein the liquid wax/solid wax mass ratio varies between 5/95 and 95/5, advantageously between 30/70 and 75/25.

9. Support according to one of claim 2, wherein the wax/polyol mass ratio varies between 95/5 and 50/50.

10. Support according to one of claim 1, wherein it further contains at least one constituent selected from the group comprising cetostearyl alcohol, sodium stearoyl lactylate and a glycerol stearate.

11. Support according to claim 1 comprising by weight:
4-50% of a wax derivative obtained by reacting jojoba wax, rice wax, candelilla wax and polyglycerol and by weight:
4-50% of sodium stearoyl lactylate,
4-50% of cetostearyl alcohol,
4-50% of glycerol stearate.

12. A method for using the support according to claim 11 as emulsifier.

13. Cosmetic composition, comprising the support according to one of claims 1.
